(19) United States
(12) Patent Application Publication
(10) Pub. No.: US 2006/0182803 A1
(43) Pub. Date: Aug. 17, 2006

(54) Oral sustained-release pharmaceutical composition of indapamide, production and use thereof

(75) Inventors: Chunyi Yeh, Chiayi County (TW); Yi-Lung Wang, Kaohsiung Hsien (TW); Ya-Sheng Yang, Hsinying City (TW); Ya-Ching Chang Chein, Kaohsiung (TW)

Correspondence Address: DELLETT & WALTERS, P. O. BOX 82788, PORTLAND, OR, 97282-0788, US

(73) Assignee: Standard Chem. & Pharm. Co., Ltd.

(21) Appl. No.: 11/060,882

(22) Filed: Feb. 17, 2005

(51) Int. Cl. A61K 9/22

(52) U.S. Cl. 424/468

(57) ABSTRACT

The present invention relates to an oral sustained-release pharmaceutical composition of indapamide and a process for producing the foregoing pharmaceutical composition. The pharmaceutical composition comprises indapamide in the amount 0.2% to 4% (w/w) of the composition, a hydrophilic polymer in the amount 10% to 30% (w/w) of the composition, a dry binding agent in the amount 2% to 20% (w/w) of the composition, and an erosion modifier in the amount 40% to 80% (w/w) of the composition. The present invention also relates to a method for delivering indapamide to a patient in need of indapamide-related therapies, which comprises administering to the patient a therapeutically effective amount of a sustained-release pharmaceutical composition in accordance with the present invention.

### BACKGROUND OF THE INVENTION

1. Field of the Invention

The present invention relates to an oral sustained-release pharmaceutical composition of indapamide, a process for producing the oral pharmaceutical composition and a method for delivering indapamide to a patient in need of indapamide-related therapies.

2. Description of Related Art

Indapamide is a sulfonamide derivative with an indole ring, which has been used as a diuretic. Indapamide inhibits the reabsorption of sodium in the cortical dilution segment and increases the urinary excretion of sodium and chloride, thereby increasing urine output and having an antihypertensive action. However, administering a conventional oral immediate-release tablet of indapamide to a patient in need of therapy rapidly increases the concentration of indapamide in the blood of the patient, and a “peak effect” occurs, which causes some undesired side effects.

U.S. Pat. No. 5,334,392 disclosed a conventional sustained-release formulation of indapamide, which comprises 1 to 2.5 mg of indapamide, polyvidone, methylhydroxyalkyl cellulose and excipients. The formulation according to U.S. Pat. No. 5,334,392 was produced by admixing about 1% of indapamide with 2-10% of polyvidone of a molecular weight between 10,000 and 700,000, sugar and colloidal silica, followed by wetting with an aqueous alcoholic solution to form granules. After drying, the granules were admixed with 30-50% of methylhydroxyalkyl cellulose with a viscosity between 1,000 and 20,000 centipoises (cps). After addition of lubricants, tablets were obtained with a hardness of 60 to 75 N as measured by diametrical crushing. However, polyvidone is so moisture-absorbing that brought less stability to the tablets produced. Furthermore, flame-proof apparatuses are required during the manufacturing process because of ethanol, which must be added to carry out the subsequent granulation. Apart from a complicated and unsafe manufacturing process, the resultant tablets unavoidably contain organic solvents as impurities.

In an attempt to the resolve the foregoing problems, WO 2004/002475 A1 disclosed a manufacturing process where the organic solvents were replaced with water. The obtained sustained-release indapamide formulation includes 1.5%-2.5% indapamide, 30-80% lactose monohydrate, 2-10% copovidon, 20-65% hypromellose and 0.1-5% lubricants. The substitute use of water for ethanol made the manufacturing process much safer. However, hypromellose renders the resultant granules so sticky that the granules cannot be mixed well with other ingredients.

To obtain a better therapeutic index at lower expenditure, there is still a need of a sustained-release pharmaceutical composition of indapamide in the an, which is produced in a safe and economical manner.

### SUMMARY OF THE INVENTION

One aspect of the present invention relates to an oral pharmaceutical composition for prolonged sustained-release of indapamide 0o the concentration of indapanilde in the blood is more stable in a period. Patients only have to take one dose of the oral pharmaceutical composition in accordance with the present invention in a 24-hour period, which decreases undesired side effects and maintains a stable concentration of indapamide in the blood to obtain a better therapeutic index.

Preferably, the oral sustained-release pharmaceutical composition of indapamide in accordance with the present invention comprises indapamide in the amount 0.2% to 4% (w/w) of the composition, a hydrophilic polymer in the amount 10% to 30% (w/w) of the composition, a dry binding agent in the amount 2% to 20% (w/w) of the composition, and an erosion modifier in the amount 40% to 80% (w/w) of the composition.

Another aspect of the present invention relates to a process to produce an oral sustained-release pharmaceutical composition of indapamide, which comprises a simple mixing in place of the complex steps of conventional wet granulation. Furthermore, granules obtainable by the process in accordance with the present invention are not sticky such that the operation and control of the subsequent procedures are much easier.

Preferably, the process in accordance with the present invention comprises mixing together indapamide in the amount 0.2% to 4% (w/w) of the composition with a hydrophilic polymer in the amount 10% to 30% (w/w) of the composition, a dry binding agent in the amount 2% to 20% (w/w) of the composition and an erosion modifier in the amount 40% to 80% (w/w) of the composition, and compressing the mixture into tablets.

Preferably, the “hydrophilic polymer” that may be employed in accordance with the present invention includes polyethylene oxide, hydroxypropyl methyl cellulose and polyvinyl alcohol. The hydrophilic polymer that may be employed in accordance with the present invention preferably has a viscosity between 20,000 and 200,000 centipoises (cps).

Preferably, the “dry binding agent” that may be employed in accordance with the present invention includes hydroxyethyl cellulose, hydroxyproxyl cellulose and pregelatinized starch.

Preferably, the “erosion modifier” that may be employed in accordance with the present invention contains at least a hydrophilic erosion accelerator, at least a hydrophobic erosion inhibitor or a combination thereof. The hydrophilic erosion accelerator is preferably, but not limited to, sugar, lactose, glucose, maltose and mannitol. The hydrophobic erosion inhibitor is preferably, but not limited to, silicate, phosphate, carbonate, glyceryl behenate and sterate.

The oral pharmaceutical composition in accordance with the present invention preferably provides a sustained release of the indapamide for about 24 hours after the composition is administered. The mixture is preferably compressed in a rotary tableting machine to obtain round-shape tablets of a diameter of about 8 mm and a hardness of about 4 to 9 kp.

More preferably, the tablets are coated with a light-resistant film. Most preferably, the light-resistant film is selected from the group consisting of hydroxypropyl methyl cellulose, polyethylene glycol and titanium dioxide.

Preferably, the oral pharmaceutical composition in accordance with the present invention may be in the form of a tablet or a capsule.

Yet another aspect of the present invention provides a method for delivering indapamide to a patient in need of indapamide-related therapies, which comprises administering to the patient a therapeutically effective amount of the sustained-release pharmaceutical composition in accordance with the present invention.

Further benefits and advantages of the present invention will become apparent after a carefull reading of the detailed description.

### BRIEF DESCRIPTION OF THE DRAWINGS

FIG. 1 is a diagram showing dissolution profiles of sustained-release indapamide formulations in accordance with the present invention, with a conventional indapamide formulation as the control group.

FIG. 2 is a diagram showing dissolution profiles of sustained-release indapamide formulations in accordance with the present invention, with a conventional indapamide formulation as the control group.

FIG. 3 is a diagram showing the profile of blood concentration of indapanide after administering a sustained-release indapamide formulation in accordance with the present invention to patients, with a conventional indapamide formulation as the control group.

### DETAILED DESCRIPTION OF THE PREFERRED EMBODIMENTS

The term “sustained release” used in accordance with the present invention refers to formulations or dosage units of indapamide that are slowly and continuously dissolved and absorbed in the stomach and gastrointestinal tract over a period of time.

Hydrophilic polymer will swell sufficiently in the gastrointestinal tract and then become gel-like. Hydrophilic polymer may be employed in accordance with the present invention includes the derivatives of cellulose, in particular the cellulose ethers such as hydroxypropyl cellulose, hydroxymethyl cellulose, methyl cellulose or methyl hydroxypropyl cellulose, and among the different commercial grades of these ethers are those showing fairly high viscosities.

Dry binding agent is used to assist the binding to the hydrophilic polymer and erosion modifier in the molding step. Dry binding agent may be employed in accordance with the present invention includes hydroxyethyl cellulose, hydroxyproxyl cellulose and pregelatinized starch.

Erosion modifier contains at least a hydrophilic erosion accelerator, at least a hydrophobic erosion inhibitor or a combination thereof. The ratio of the hydrophilic erosion accelerator and the hydrophobic erosion inhibitor control the rate of sustained release.

The oral sustained-release pharmaceutical composition of indapamide in accordance with the present invention provides prolonged sustained release of indapamide so the concentration of indapamide in the blood is more stable.

Therefore, patients in need of indapamide-related therapies only have to take one dose of the pharmaceutical composition in accordance with the present invention in a 24-hour period, which decreases undesired side effects and sustains a stable concentration of indapamide in the blood.

### EXAMPLES

### Example 1

### Sustained-Release Indapamide Formulation (1) and a Process for the Production Thereof

1. 1 Sustained-Release Indapamide Formulation (1)

| Composition | Amount (mg) |
|---|---|
| Indapamide | 1.5 |
| Lactose | 112.5 |
| Dicalcium phosphate | 30 |
| Hydroxypropyl methyl cellulose (100,000 cps) | 40 |
| Hydroxyproxyl cellulose | 10 |
| Silicon dioxide | 3 |
| Magnesium stearate | 3 |

1.2 Steps

Step 1: Indapamide, lactose, dicalcium phosphate, hydroxypropyl methyl cellulose, hydroxyproxyl cellulose, silicon dioxide and magnesium sterate were mixed well.

Step 2: The mixture in step 1 was compressed in a rotary tableting machine to obtain tables having a diameter of approximately 8 mm and a hardness of approximately 4 to 9 kp.

### Example 2

### Sustained-Release Indapamide Formulation (2) and a Process for the Production Thereof

2.1 Sustained-Release Indapamide Formulation (2)

| Composition | Amount (mg) |
|---|---|
| Indapamide | 1.5 |
| Lactose | 72.5 |
| Dicalcium phosphate | 80 |
| Hydroxypropyl methyl cellulose (100,000 cps) | 30 |
| Hydroxyproxyl cellulose | 10 |
| Silicon dioxide | 3 |
| Magnesium stearate | 3 |

2.2 Steps

Step 1. Indapamide, lactose, dicalcium phosphate, hydroxypropyl methyl cellulose, hydroxyproxyl cellulose, silicon dioxide and magnesium sterate were mixed well.

Step 2: The mixture in step 1 was compressed in a rotary tableting machine to obtain tables having a diameter of approximately 8 mm and a hardness of approximately 4 to 9 kp.

### Example 3

### Sustained-Release Indapamide Formulation (3) and Method for the Production Thereof

3.1 Sustained-Release Indapamide Formulation (3)

| Composition | Amount (mg) |
|---|---|
| Indapamide | 1.5 |
| Lactose | 115.0 |
| Hydroxypropyl methyl cellulose (100,000 cps) | 47.5 |
| Pregelatinized starch | 30.0 |
| Silicon dioxide | 3.0 |
| Magnesium stearate | 3.0 |

3.2 Steps

Step 1: Indapamide, lactose, hydroxypropyl methyl cellulose, starch, silicon dioxide and magnesium sterate were mixed well.

Step 2: The mixture in step 1 was compressed in a rotary tableting machine to obtain tables having a diameter of approximately 8 mm and a hardness of approximately 4 to 9 kp.

### Example 4

### Sustained-Release Indapamide Formulation (4) and Method for the Production Thereof

4.1 Sustained-Release Indapamide Formulation (4)

| Composition | Amount (mg) |
|---|---|
| Indapamide | 1.5 |
| Mannitol | 58.5 |
| Dicalcium phosphate | 60 |
| Hydroxypropyl methyl cellulose (30,000 cps) | 58 |
| Hydroxyproxyl cellulose | 16 |
| Silicon dioxide | 3 |
| Magnesium stearate | 3 |

4.2 Steps

Step 1: Indapamide, mannitol, dicalcium phosphate, hydroxypropyl methyl cellulose, hydroxyproxyl cellulose, silicon dioxide and magnesium sterate were mixed well.

Step 2: The mixture in step 1 was compressed in a rotary tableting machine to obtain tables having a diameter of approximately 8 mm and a hardness of approximately 4 to 9 kp.

### Example 5

### Sustained-Release Indapamide Formulation (5) and Method for the Production Thereof

5.1 Sustained-Release Indapamide Formulation (5)

| Composition | Amount (mg) |
|---|---|
| Indapamide | 1.5 |
| Lactose | 132.5 |
| Dicalcium phosphate | 2.5 |
| Hydroxypropyl methyl cellulose (100,000 cps) | 47.5 |
| Hydroxyproxyl cellulose | 10 |
| Silicon dioxide | 3 |
| Magnesium stearate | 3 |

5.2 Steps

Step 1: Indapamide, lactose, dicalcium phosphate, hydroxypropyl methyl cellulose, hydroxyproxyl cellulose, silicon dioxide and magnesium sterate were mixed well.

Step 2: The mixture in step 1 was compressed in a rotary tableting machine to obtain tables having a diameter of approximately 8 mm and a hardness of approximately 4 to 9 kp.

Step 3: Tablets in step 2 were coated film with 4.0 mg hydroxypropyl methyl cellulose, 0.75 mg polyethylene glycol and 0.25 mg titanium dioxide.

### Example 6

### Ingredient-Releasing Rate Test

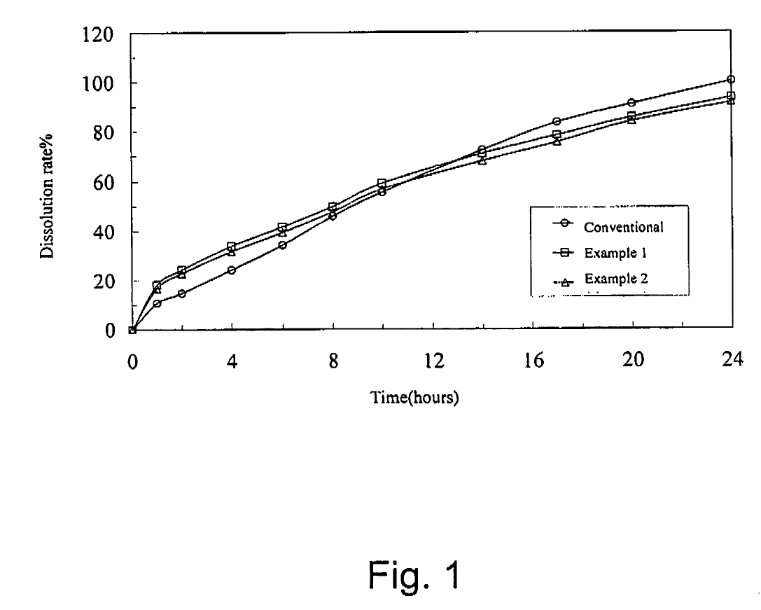

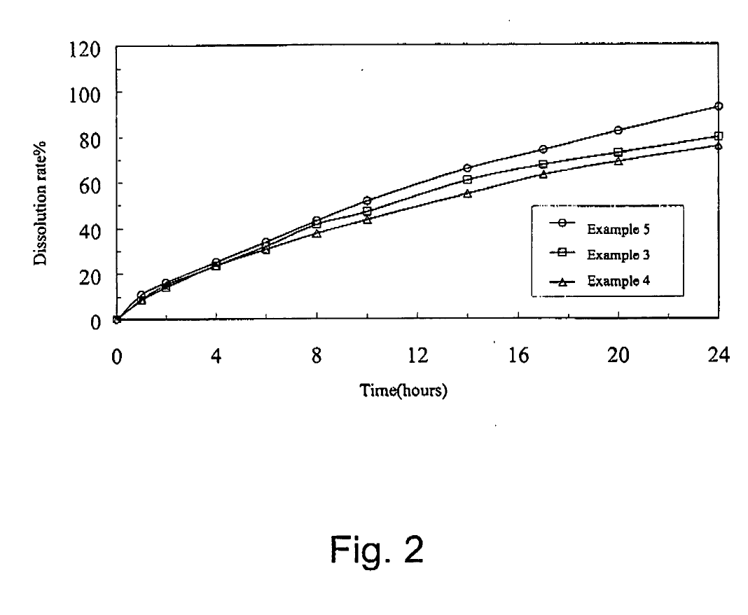

The rates of indapamide releases from the indapamide formulations made according to Examples 1-5 and a conventional indapamide that had an equal quantity of indapamide were evaluated in a dissolution test in accordance with instructions in the United States Pharmacopoeia (U.S.P) 27^{th} edition. In this test, each indapamide formulation and 500 mL of pH 6.8 phosphate buffer were poured into a vessel and heated to 37±0.5° C. Then, the mixture was stirred in a mixer at 75 rpm. The results are provided in Table 1 and FIGS. 1 and 2.

TABLE 1
Dissolution rate (%)

| Time | | | | | | |
|---|---|---|---|---|---|---|
| (hours) | Conventional | Example 1 | Example 2 | Example 3 | Example 4 | Example 5 |
| 0 | 0 | 0 | 0 | 0 | 0 | 0 |
| 1 | 10.68 | 18.06 | 16.62 | 8.43 | 8.73 | 10.90 |
| 2 | 14.67 | 24.24 | 22.71 | 13.95 | 15.14 | 16.00 |
| 4 | 24.22 | 33.82 | 31.68 | 23.46 | 23.38 | 24.92 |
| 6 | 34.28 | 41.70 | 39.43 | 31.95 | 30.65 | 33.79 |
| 8 | 45.92 | 49.90 | 47.72 | 41.55 | 37.63 | 43.05 |
| 10 | 55.54 | 59.05 | 56.80 | 46.93 | 43.55 | 51.48 |
| 14 | 72.05 | 70.75 | 67.81 | 60.44 | 54.63 | 65.60 |
| 17 | 83.28 | 78.03 | 75.25 | 67.39 | 62.82 | 73.97 |
| 20 | 90.78 | 85.33 | 83.69 | 72.55 | 68.77 | 82.19 |
| 24 | 100.0 | 93.33 | 91.36 | 79.50 | 75.50 | 92.69 |

The results in Table 1 and the data in FIGS. 1 and 2 show that all the indapamide formulations have stable sustained release efficacy in the pH 6.8 phosphate buffer. The ratios of the indapamide released from the formulations achieved 75% after 24 hours.

### Example 7

### The Blood Concentration of Sustained-Release Indapamide

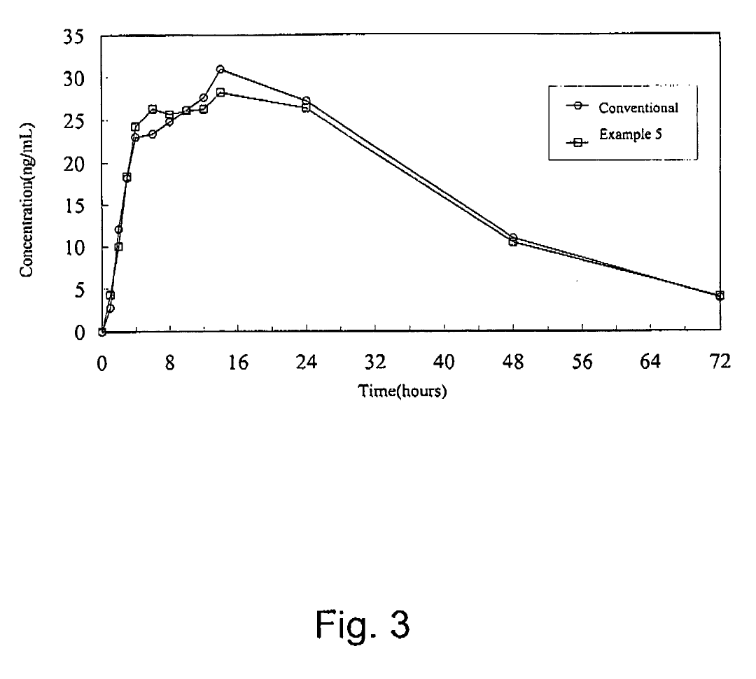

The blood concentration of indapamide was measured in five patients after taking tablets containing 1.5 mg indapamide in example 5 and conventional indapamide tablets. The data shown in Table 2 and FIG. 3 demonstrate that indapamide formulations in accordance with the present invention can be controlled-release for more than 24 hours.

TABLE 2
The blood concentrations of indapamide (ng/mL)

| Time(Hours) | Conventional | Example 5 |
|---|---|---|
| 0 | 0 | 0 |
| 1.0 | 2.79 | 4.30 |
| 2.0 | 12.12 | 10.01 |
| 3.0 | 18.20 | 18.38 |
| 4.0 | 23.00 | 24.22 |
| 6.0 | 23.38 | 26.30 |
| 8.0 | 24.78 | 25.66 |
| 10.0 | 26.16 | 26.06 |
| 12.0 | 27.64 | 26.26 |
| 14.0 | 30.98 | 28.24 |
| 24.0 | 27.16 | 26.32 |
| 48.0 | 10.98 | 10.44 |
| 72.0 | 3.87 | 4.02 |

Accordingly, advantages of the sustained-release indapamide formulations in accordance with the present invention include the following.
1. Patients can reduce the frequency of ingesting such medicine.
2. The blood concentration of indapamide fluctuates less so that antinociceptive tolerance of patients is promoted.

Although the invention has been explained in relation to its preferred embodiment, many other possible modifications and variations can be made without departing from the spirit and scope of the invention as hereinafter claimed.

What is claimed is:

1. An oral sustained-release pharmaceutical composition of indapamide comprising:
indapamide in the amount 0.2% to 4% (w/w) of the composition,
a hydrophilic polymer in the amount 10% to 30% (w/w) of the composition,
a dry binding agent in the amount 2% to 20% (wlw) of the composition, and
an erosion modifier in the amount 40% to 80% (w/w) of the composition.

2. The oral pharmaceutical composition as claimed in claim 1, wherein the hydrophilic polymer is selected from the group consisting of polyethylene oxide, hydroxypropyl methyl cellulose and polyvinyl alcohol.

3. The oral pharmaceutical composition as claimed in claim 1, wherein the hydrophilic polymer has a viscosity between 20,000 and 200,000 centipoises (cps).

4. The oral pharmaceutical composition as claimed in claim 1, wherein the dry binding agent is selected from the group consisting of hydroxyethyl cellulose, hydroxyproxyl cellulose and pregelatinized starch.

5. The oral pharmaceutical composition as claimed in claim 1, wherein the erosion modifier contains at least a hydrophilic erosion accelerator, at least a hydrophobic erosion inhibitor or a combination thereof:

6. The oral pharmaceutical composition as claimed in claim 5, wherein the hydrophilic erosion accelerator is selected from the group consisting of sugar, lactose, glucose, maltose and mannitol.

7. The oral pharmaceutical composition as claimed in claim 5, wherein the hydrophobic erosion inhibitor is selected from the group consisting of silicate, phosphate, carbonate, glyceryl behenate and sterate.

8. The oral pharmaceutical composition as claimed in claim 1, which provides a sustained release of the indapamide for about 24 hours after the composition is administered.

9. The oral pharmaceutical composition as claimed in claim 1, which further contains a light-resistant coating.

10. The oral pharmaceutical composition as claimed in claim 1, which is in the form of a tablet or a capsule.

11. A process for producing an oral sustained-release pharmaceutical composition of indapamide comprising
mixing together indapamide in the amount 0.2% to 4% (w/w) of the composition with a hydrophilic polymer in the amount 10% to 30% (w/w) of the composition, a dry binding agent in the amount 2% to 20% (w/w) of the composition and an erosion modifier in the amount 40% to 80% (w/w) of the composition, and
compressing the mixture into tablets.

12. The process as claimed in claim 11, wherein the hydrophilic polymer is selected from the group consisting of polyethylene oxide, hydroxypropyl methyl cellulose and polyvinyl alcohol.

13. The process as claimed in claim 11, wherein the hydrophilic polymer has a viscosity between 20,000 and 200,000 centipoises (cps).

14. The process as claimed in claim 11, wherein the dry binding agent is selected from the group consisting of hydroxyethyl cellulose, hydroxyproxyl cellulose and pregelatinized starch.

15. The process as claimed in claim 11, wherein the erosion modifier contains at least a hydrophilic erosion accelerator, at least a hydrophobic erosion inhibitor or a combination thereof.

16. The process as claimed in claim 15, wherein the hydrophilic erosion accelerator is selected from the group consisting of sugar, lactose, glucose, maltose and mannitol.

17. The process as claimed in claim 15, wherein the hydrophobic erosion inhibitor is selected from the group consisting of silicate, phosphate, carbonate, glyceryl behenate and sterate.

18. The process as claimed in claim 11, further comprising coating the tablets with a light-resistant film.

19. The process as claimed in claim 18, wherein the light-resistant film is selected from the group consisting of hydroxypropyl methyl cellulose, polyethylene glycol and titanium dioxide.

20. The process as claimed in claim 11, wherein the mixture is compressed in a rotary tableting machine to obtain a round-shape tablet that has a diameter of about 8 mm and a hardness of about 4 to 9 kp.

21. A method for delivering indapamide to a patient in need of indapamide-related therapies, which comprises administering to the patient a therapeutically effective amount of a sustained-release pharmaceutical composition as claimed in claim 1.
